(19) United States
(12) Patent Application Publication
(10) Pub. No.: US 2015/0272775 A1
(43) Pub. Date: Oct. 1, 2015

(54) MULTI-PHASE MIXED MATERIAL THERAPY PACK

(71) Applicants: Eric Fayez CHEHAB, Mountain View, CA (US); Brian J. FAHEY, Palo Alto, CA (US); Timothy MACHOLD, Moss Beach, CA (US); Zachary J. MALCHANO, San Francisco, CA (US); Curtis TOM, San Mateo, CA (US); Steven H. TREBOTICH, Newark, CA (US)

(72) Inventors: Eric Fayez CHEHAB, Mountain View, CA (US); Brian J. FAHEY, Palo Alto, CA (US); Timothy MACHOLD, Moss Beach, CA (US); Zachary J. MALCHANO, San Francisco, CA (US); Curtis TOM, San Mateo, CA (US); Steven H. TREBOTICH, Newark, CA (US)

(21) Appl. No.: 14/673,520

(22) Filed: Mar. 30, 2015

Related U.S. Application Data
(60) Provisional application No. 61/973,741, filed on Apr. 1, 2014.

(51) Int. Cl. A61F 7/03

(52) U.S. Cl. CPC A61F 7/03; A61F 2007/0292

(57) ABSTRACT

Thermal packs adapted for use in a muscle stimulation system, comprising an external housing with a first material therein and at least one phase change element therein.

### CROSS-REFERENCE TO RELATED APPLICATION

This application claims the benefit of U.S. Provisional Application No. 61/973,741, filed Apr. 1, 2014, incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

Cold packs, heat packs, ice packs, and similar products are commonly-used and well-described in the prior art as therapeutic technologies having a host of benefits. These include pain relief, reduction in local swelling, and assistance with certain aspects of medical care. As such these products have gained widespread use and have numerous applications in emergency medicine, sports medicine, home first-aid, and in other areas of healthcare. There are also numerous industrial applications for cold packs and similar technologies, for example in the food preparation and shipping/transit industries. In many cases, existing technologies that have been described in the prior art perform suitably for the desired applications.

For certain advanced applications, however, existing technologies described in the prior art fail to fulfill all required performance prerequisites, and as such are ineffective or perform sub-optimally. Shortcomings may take many forms. For example, existing cold packs may have improper mechanical features, such as flexibility, conformability, etc. Existing packs may also have thermal properties that are not desirable: not reaching appropriate temperatures, not staying at appropriate temperatures for the desired period of time, not having a suitable temperature distribution, etc. One skilled in the art will note that the limitations mentioned herein are simply representative of the types of limitations of existing technologies, and by no means represents an exhaustive list.

Advanced applications, especially but not limited to when thermal packs are applied to human or animal bodies, may require novel solutions for technology to be effective. For example, there are applications where the following features may be desired: (a) cools tissue to a temperature very close to skin freezing temperature (ex. 32-36 degrees Fahrenheit), (b) causes tissue to reach this temperature and hold at this temperature without over-cooling (i.e., not over-shooting the target temperature), (c) causes tissue to reach this target temperature very quickly, (d) holds the desired temperature for an extended period of time, (e) stays mechanically conformable to a curved surface of the body when at the desired usage temperature, (f) can apply thermal energy across a wide surface area without ‘gaps’ or regions of poor contact/thermal transfer, and (g) can be regenerated (i.e., pack is re-cooled and ready for another use) in a short period of time (ex. 1-3 hours) into a form that is ready for re-use. As the following paragraphs will illustrate, existing cold pack technologies described in the prior art may achieve a subset of these requirements, but do not provide all of the desired attributes. As such, for this example application and similar ones, novel technologies are needed to achieve desired medical treatment or other performance goals.

With regard to the requirements listed above, (a) and (b) are particularly important because they are related to subject safety and can be difficult to achieve in tandem. Failing to meet requirement (b) is especially undesirable in light of requirement (a), and any potential solution that did not satisfy this criteria would immediately be disqualified from use. In order to err on the side of caution, to have broader appeal across less specialized applications, and/or for other reasons, most existing technologies do not attempt to meet requirement (a). Those solutions that are capable of meeting requirement (a) fall short of multiple other requirements listed above.

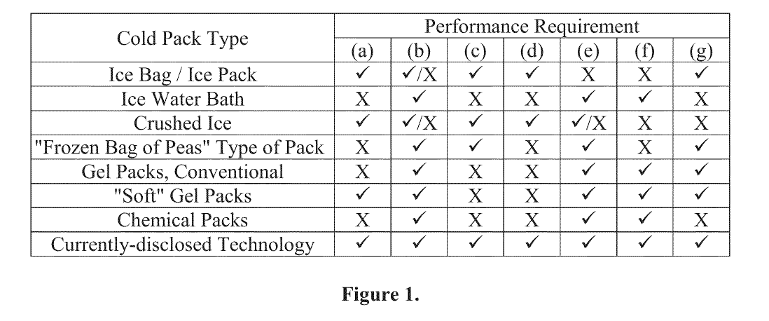

No thermal pack technologies described in the prior art meet all of the requirements listed above. For example, ice packs (frozen water bag or similar) are rigid and fail to achieve the conformability requirement (e), which limits utility to body parts that have essentially no radius of curvature. Depending on how they are stored, they also may be too cold for use, thus failing criteria (b) as well. Ice water baths in practical sizes generally fail to achieve requirements (a) and/or (d). Crushed-ice or “frozen bag of peas” type configurations have non-continuous structural arrangements that lead to gaps in surface contact, thus failing requirement (f). Crushed ice configurations also fail requirement (g), as once they melt during use, they cannot be easily regenerated (i.e. re-freeze) without becoming a solid block of ice which fails to meet the conformability requirement (e). Gel packs, even advanced “soft-gel” packs, cannot meet all of requirements (a)-(d) simultaneously, especially in light of requirement (e). Instant or chemical-based packs (such as those created by breaking an internal water lumen, causing the water to mix with a chemical such as urea or ammonium nitrate, will fail requirements (a) and (d) due to available thermal energy and will fail requirements (f) and (g) due to their single-use nature. A number of these examples are summarized in FIG. 1.

As intended usage moves from simple applications to those with more advanced/precise requirements, existing thermal pack technologies fail to be suitable. Novel thermal pack technologies are needed in order to meet all necessary performance requirements for certain applications in the medical field and in other industries. These novel technologies need to address the shortcomings of technologies described in the prior art in order to better enable direct or indirect medical treatments or to assist with other processes. Technologies that achieve these goals will also provide improved products for more simple applications, as well. Disclosed within are devices, systems, and methods that address deficiencies in the prior art and achieve these goals.

### SUMMARY OF THE INVENTION

Detailed within are devices, systems, and methods for improved thermal pack technology that address shortcomings associated with the prior art. Several embodiments and implementations of the invention are described herein, though it will be evident to those skilled in the art that these are exemplary and that numerous configurations of the present invention are possible. An important aspect of many of the embodiments of the present invention is that the systems, devices, and methods described simultaneously achieve all of the performance criteria outlined in the Background section of this disclosure. Variation embodiments may achieve only a subset of the outlined performance criteria in exchange for improved efficacy in other key performance areas. It will be clear to those skilled in the art that the presently-disclosed thermal pack technology has applications in other industries aside from medicine, and that variations on the same concept that utilize different target temperatures would also constitute novel embodiments of technologies with profound industrial uses. While the present disclosure focuses on packs configured to provide cold therapy, it will be clear to those skilled in the art that variation embodiments can be configured to provide warm or hot therapy with no loss of novelty. It should be appreciated that different aspects of the invention can be appreciated individually, collectively, or in combination with each other.

Preferable embodiments of the devices and systems are constructed of multiple constituent materials with different thermal and mechanical properties. These materials are encapsulated in an outer bag or pouch, which contacts the desired application surface (for example, the tissue of a patient or a compartment in a shipping container). In some embodiments, an additional thin sheath or covering material may be placed around the outer bag or pouch prior to use. This covering material may be disposable or reusable. For example, in a hospital environment, the covering material may be placed around a cold pack prior to use on a patient, then removed and discarded before the cold pack is returned to a cooling chamber for regeneration. This process may help with infection control to allow a single pack to be applied to multiple patients, and also have additional benefits.

In preferable embodiments, one material contained within the thermal pack pouch is a gel-type material. In preferable embodiments, this gel material has thermal properties such that it's freezing point is colder than the freezing point of water. In some embodiments, the freezing point is much colder than the freezing point of water. This material property will allow the presently-described cold pack to remain soft and flexible and conformable around a curved surface even at low temperatures. In variation embodiments, a liquid material or other suitable material known to those skilled in the art may be substituted for the gel-like material.

In preferable embodiments, the thermal packs also contain small packets or pods of a second material (which in this disclosure may be referred to as a phase change material). In preferable embodiments this phase change material will be a liquid at room temperature and have a freezing point warmer than that of the gel material used. In some embodiments, the freezing point of the second material is close to the freezing point of water. The packets or pods that contain the units of phase change material are composed of a thin or otherwise thermally-conductive material so that they may transfer thermal energy to the gel material that is also found in the bag or pouch that comprises the thermal pack. In some embodiments, more than one material may be contained in the packets or pods, or multiple types or shapes/sizes of pods or packets may be used. In preferable embodiments, packets or pods of this secondary material are of such a size, shape, and distribution such that the mechanical conformability of the cold pack is not compromised, even when the material contained within the pods or packets is frozen.

In a preferable method of use, the novel cold pack is stored in a freezer or other suitable cooling chamber at a temperature such that the material(s) in the pods/packets freezes (i.e. undergoes a phase change). This phase change results in a large amount of stored thermal energy in the packets within the cold pack. In preferable methods of use, the storage temperature chosen for use also allows the gel material within the cold pack to remain soft and flexible, even when the phase change materials have frozen. As such, during use, the cold pack remains conformable around curved surfaces (for example, around the surface of an arm or a leg) with even, continuous contact to the intended surface. During use, the gel material acts as a contact medium to evenly distribute energy to the intended surface. However, it is known that, when used stand-alone, the thermal energy within a gel material will exhaust quickly. In the present invention, thermal energy is continually transferred from the phase-change (i.e., frozen) material in the packets or pods to the gel material that the packets/pods are embedded in. As such, the cold pack in the current invention will exhaust very slowly relative to standard gel packs, while maintaining all of the mechanical and conformability benefits of gel packs described in the prior art.

In preferable embodiments of the method, the presently-disclosed gel packs can be easily re-used by being placed back into a freezer or equivalently suitable cooling chamber. Since the phase change material is contained within packets or pods, it will not mix with the gel material once it has changed from solid to liquid phase (i.e., melted). Also, the packets or pods may be used to maintain the desired shape that the phase change material pieces will take once they are frozen.

In preferable embodiments, including many applications in the fields of medicine and healthcare, phase change materials will be chosen that have a freezing point close to the freezing point of water. This phase change transition point will be a dominant factor in dictating the temperature that the cold pack maintains over an extended period of time. In variation embodiments, alternate phase change materials may be selected for use which guide the cold pack to a different temperature. In alternate configurations, similar strategies may be used to configure the disclosed devices and systems as a heat or hot pack intended to deliver hot therapy or to apply warmth to an object for extended periods of time.

Preferable embodiments of the presently-disclosed technology achieve all of the performance requirements outlined above in the Background section of this disclosure and have additional positive attributes that are not listed. As such, these devices, systems, and methods provide clear useful benefits. For example, they allow for safe, effective, long-lasting, low temperature cold therapy to be provided to the body evenly around a surface with a radius of curvature. No other technologies described in the prior art can achieve all of these goals simultaneously. In addition, the presently-disclosed technology is easily reusable, which is an added benefit that makes it a practical, low-cost choice for therapy.

### BRIEF SUMMARY OF THE DRAWINGS

As shown in FIG. 1, a summary of performance attributes and properties of cold packs disclosed in the prior art compared to those associated with the presently disclosed technology.

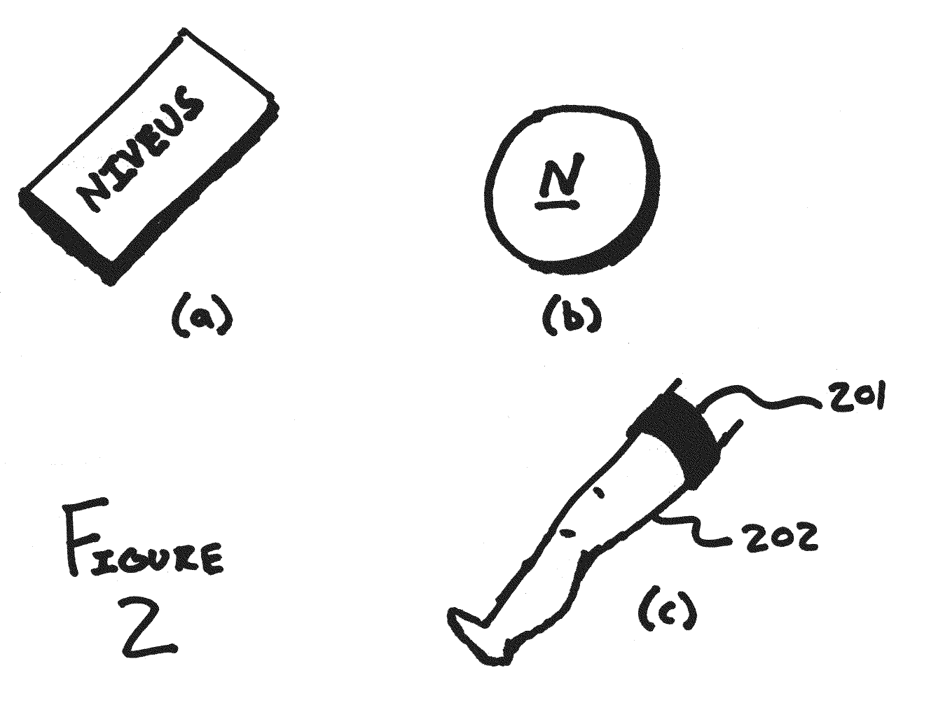

As shown in FIG. 2, preferable embodiments of configurations of a thermal pack and a illustrative example regarding a method of use.

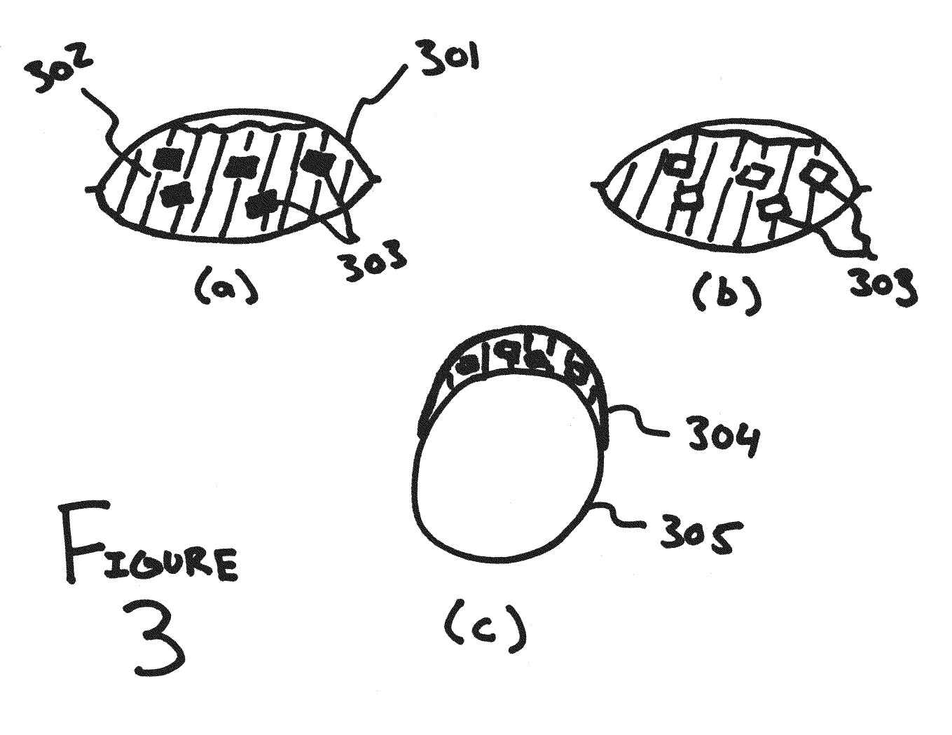

As shown in FIG. 3, cross-sectional views of a preferable embodiment of devices and systems at multiple temperatures and in multiple mechanical configurations.

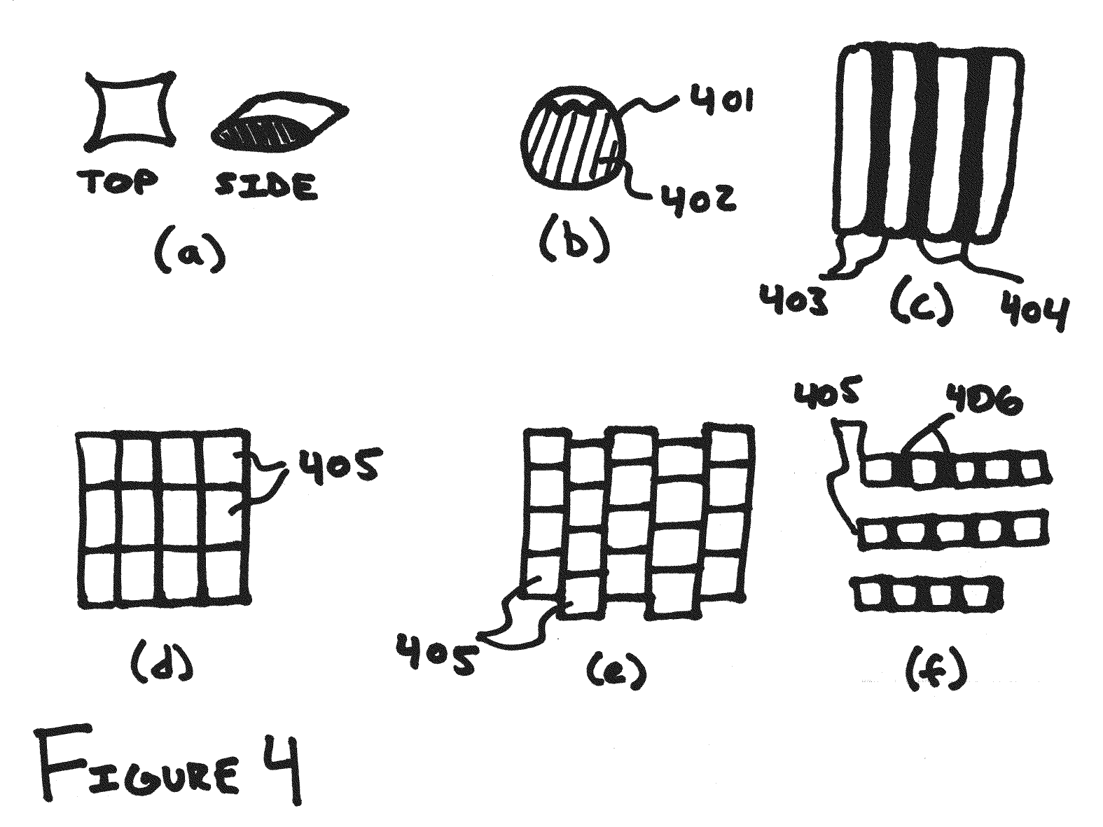

As shown in FIG. 4, variations of preferable embodiments of devices and systems used to contain and arrange phase change materials.

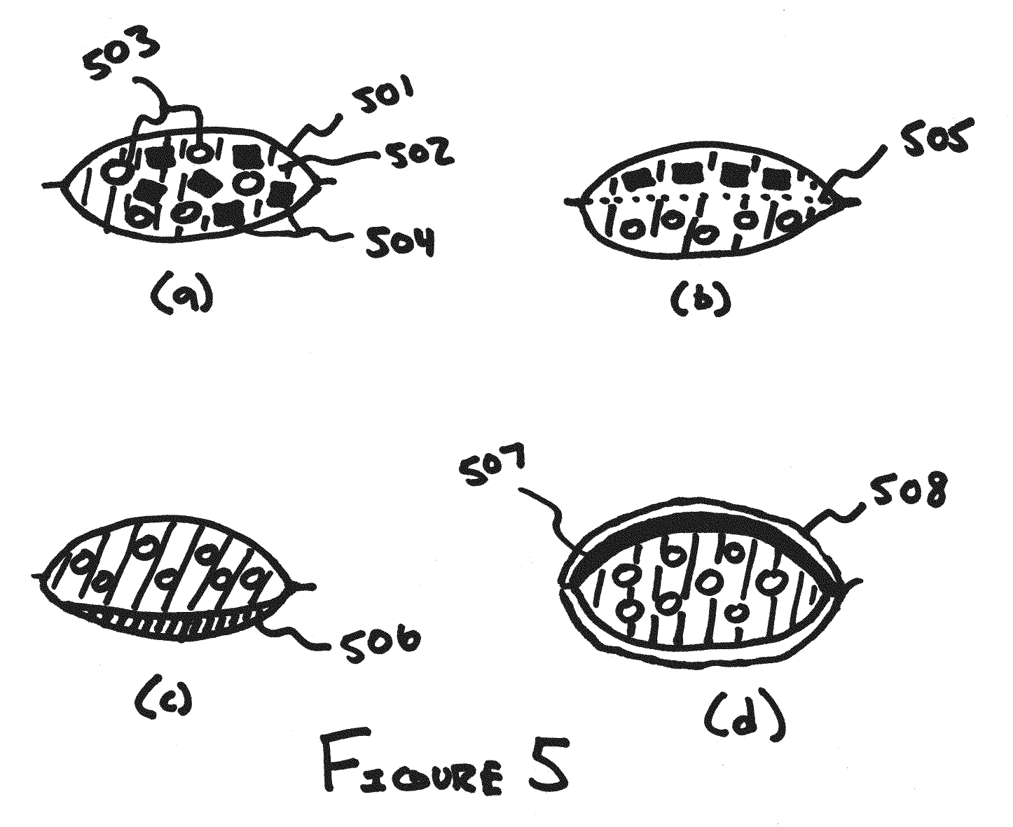

As shown in FIG. 5, cross-sectional views of variation embodiments of the devices and systems shown in multiple configurations.

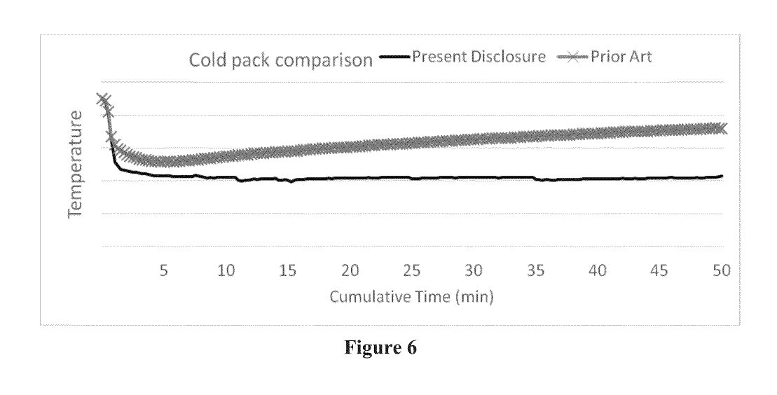

As shown in FIG. 6, performance data of a preferred embodiment of the presently disclosed devices and systems vs. a leading commercially-available cold pack described in the prior art.

### DETAILED DESCRIPTION OF THE INVENTION

The invention provides methods, devices, and systems for improved thermal pack technology. Though this disclosure uses cold pack technologies as an illustrative example, those skilled in the art will appreciate that the presently-disclosed invention may be applied with utility to warm or hot pack therapies as well. Various aspects of the invention described herein may be applied to any of the particular applications set forth below or for any other types of thermal pack applications across various industries. The invention may be applied as a standalone device, or as part of an integrated system, such as a medical treatment system. It shall be understood that different aspects of the invention can be appreciated individually, collectively, or in combination with each other.

With reference to FIG. 2, thermal packs described herein may take on a variety of general shapes or configurations, such as the rectangular configuration shown in (a) or the circular/ovular configuration shown in (b). Regardless of shape, preferable embodiments will retain the ability to conform about a curved surface when at the desired use temperature. This concept is illustrated in FIG. 2(c), where the thermal pack 201 conforms around the curved surface of the leg 202. In variation implementations of the preferred embodiment, the thermal packs are ‘donut shaped’ and can be placed around a desired application area for circumferentially-applied thermal therapy. In variations of this embodiment, thermal packs are rectangular but are manually wrapped circumferentially completely or nearly completely around a curved surface. In preferable embodiments, thermal packs may effectively conform about a curved surface without meaningful gaps or ‘wrinkle-pockets’ that are created by the mechanical properties of the pack or its constituent materials. As an illustrative example, some packs described in the prior art are configured to have linear mechanical stress points such that when they are forced to bend along a radius of curvature, portions on the pack buckle outward away from the desired contact surface. This creates gaps in the applied cooling contact area that lead to non-ideal discontinuous thermal coverage which can render packs unsuitable for many desired applications. Preferable embodiments will have no large gaps in the cooling contact surface and a low number of incidental areas of discontinuous contact. This may be contrasted with crushed ice or ‘frozen bag of peas’ style thermal packs, which by nature have a vast number of small sized contact gaps which in the aggregate substantially impact thermal performance and have secondary effects related to ‘pathways’ or ‘canals’ that exist across the entire region of intended cooling.

Preferable embodiments of the devices and systems incorporate the use of an outer pouch or enclosure material which holds other thermal pack components. This pouch is generally soft, flexible, and strong enough for multiple uses without breaking or leaking. In some embodiments, this outer pouch is biocompatible and may be disinfected with commonly-used cleaning agents without breaking down. In preferable embodiments this material can be sealed using heat-sealing techniques or other suitable alternative closure methods known to those skilled in the art. For many embodiments, one suitable material may be a low density polyethylene material. In some embodiments, hybrid multi-material composites or laminates may be used to balance multiple material properties, such as flexibility, tear strength, and/or moisture control. These composites may employ the use of materials such as polyethylene terephthalate (PET), nylon, or other suitable materials known to those skilled in the art. Preferable embodiments will utilize thin pouch layers to facilitate thermal transfer to desired contact surfaces, for example materials with thicknesses in the 0.0005 inches to 0.020 inches range. In some embodiments, the outer layer of the thermal pack may contain a means for attaching or mating with a target location, such as tape, elastic rings, bands, or other means known to those skilled in the art.

A key aspect of the presently-disclosed devices and systems is the use of multiple constituent materials housed within this outer pouch of the thermal pack. In preferable embodiments, one of these materials is a gel-type material. Gel materials may be composed of soft hydrogels, such as those well-known to those skilled in the art. Preferable embodiments may utilize hydrogel mixtures, such as those which include salts, fibers, sand, or other additives that give the gel an appropriate consistency and/or reduce the freezing temperature of the gel to a lower value. Preferable embodiments will utilize gel solutions that have a freezing point that is lower than the freezing point of water, such that the gel material remains soft and flexible even at or below a temperature of 32 degrees Fahrenheit. Some implementations may use gel materials that remain soft and flexible at a temperature of 0 degrees Fahrenheit, −15 degrees Fahrenheit, or colder. Variations of the preferred embodiments may substitute fluid (such as salted water, or equivalent), fluid mixtures (ex. fluid and sand, or equivalent), foam-type, or other suitable materials known to those skilled in the art for the gel type material.

In some implementations of preferable embodiments, additional additives may be mixed into the gel-type material used in the thermal pack in order to improve thermal conductivity and/or thermal transfer. As one illustrative example, aluminum powder or other materials may be added to the gel material to improve thermal exchange between constituent components of the thermal pack and/or between the thermal pack and the contact surface. Those skilled in the art will recognize that other additive materials that are commercially-available may be substituted for aluminum powder with no loss of novelty.

In preferable embodiments, the thermal packs also contain small packets or pods of a second material (which in this disclosure may be referred to as a phase change material). These packets or pods of phase change material are suspended in or otherwise distributed among the gel material within the thermal pack pouch. Preferable embodiments of the devices and systems will utilize a phase change material that is a liquid at room temperature and has a freezing point warmer than that of the gel material used. Some implementations of preferable embodiments will use a phase change material with a freezing point close to the freezing point of water. In variation embodiments, when the thermal pack is used for heating, the phase change material may be characterized by its melting point which may be much higher than the freezing point of water.

FIG. 3 assists in describing the functionality of preferable embodiments of the devices, systems, and methods of use associated with the thermal packs. In (a), a thermal pack at room temperature is shown. The outer pouch 301 contains both the gel material 302 and packets of phase change material 303, which at this temperature remain in an unfrozen or liquid phase. The material comprising the packets prevents the liquid-state phase change material from mixing with the gel-type material. In (b), shown is the same thermal pack as in (a) but now at a temperature below the freezing point of water but above the freezing point of the gel—i.e., a desirable usage temperature for a thermal pack configured to be used as a cold pack. In this scenario, the phase change material within the packets 303 is frozen in the solid phase while the gel material remains soft and flexible. In (c), the cold pack 304 is applied to a surface with a radius of curvature 305. Despite the frozen nature of the phase change material, the cold pack conforms around the curved contact surface and makes even contact without meaningful gaps or zones of material buckling.

In preferable embodiments, the phase change material is contained in a packet or pod constructed of a material that facilitates the transmission of thermal energy from the phase change material to the gel material in the pack. Without wishing to be bound by any theory, it is believed that the overall efficacy of the disclosed thermal packs will be impacted by the ability for the phase change materials to effectively transfer and distribute thermal energy to the gel material. Many materials may be suitable to contain phase change material, including plastics such as low density polyethylene, metals such as aluminum, and other materials known to those skilled in the art. The optimal packet material of choice will depend on the precise implementation of the preferred embodiment chosen for use, and may be influenced by factors such as cost, weight, flexibility, stability, and rigidity in addition to thermal transfer factors.

Preferable embodiments of the devices, systems, and methods will configure the design, storage, and method of use of the thermal packs such that sufficient energy may be transferred to hold a contact surface at or near the desired target temperature for 30-60 minutes or longer. Various implementations of these preferred embodiments may be designed to last for longer or shorter times. The relative volume of phase change and gel materials contained within the thermal packs may be selected to balance both thermal and mechanical performance. In some embodiments, gel material and phase change material may be added to the pack in amounts of equal weight. In variation embodiments, a fixed number of phase change material packets may be added to the pack, this number depending on the size and shape of packets used. In further variation embodiments, the number of packets may be large and the volume of phase change material may far exceed the volume of gel material as the size of packets becomes very small.

With reference to FIG. 4, different implementations of preferred embodiments may utilize different configurations and/or shapes of phase change material packets or pods. In (a), a simple rectangular or pillow-shaped quasi-cube design is shown from both top and side/oblique views. Some implementations may use packets of this shape. Packets may range in size from very small (0.1 inches square) to larger (3 inches square) depending on usage, manufacturing, and other considerations. As shown in (b), a cross-sectional view of a spherical or nearly-spherical design for a pod 401 containing phase change material 402 may also be used. Preferable embodiments that implement packet designs shown in (a) or (b) (or similar) may use many packets (example: 15-75 packets) distributed randomly or semi-randomly in the gel material of the thermal pack. Depending on size and shape, in preferable embodiments each packet may contain between 0.5-20 ml of phase change material.

In variation embodiments, it may be desirable to have the phase change material packets assume a more orderly configuration relative to one another. This may be desirable for performance, manufacturing, ease of use, other reasons, or some combination thereof. With reference to FIG. 4, in (c) elongated rows of phase change material packets 403 are attached to one another by elongated bonded sections 404. These bonded sections may be made from the same material (either continuous or attached) as the phase change packets or of another suitable material. In (d) and (e), phase change material packets 405 are arranged symmetric and asymmetric grids, respectfully. In (f), elongated strands of phase change packets 405 are created with intermittent separating layers 406. In various implementations of thermal pack embodiments, a single or multiple grid of phase change packets or pods may be used, or a combination of several types of grid/packet/strand/pod may be utilized. It should be understood that the various aspects of the examples provided may be used in combination with one another. It will also be clear to those skilled in the art that examples provided in FIG. 4 are shown for illustrative purposes only and that many configurations are possible.

When thermal packs are configured as cold packs, preferable embodiments of the devices and systems will utilize phase change materials that freeze at a temperature warmer than the freezing temperature of the gel material. Specific materials for use may vary depending on the precise desired phase change temperature required, which may be dictated by the intended application of the cold pack. This is one major advantage of the presently disclosed devices and systems: with minor material changes the target pack temperature can be varied while maintaining the ability to achieve all of the desired attributes specified in FIG. 1. As an illustrative example, for many human-use applications when the cold pack will be applied to tissue, a phase change material that freezes at or near 32 degrees Fahrenheit may be desirable for use. If the cold pack is intended to be applied across an insulating layer, a colder phase change material may be desired to achieve similar degrees of tissue cooling. Many phase change materials are commercially-available and well known to those skilled in the art. Many of these materials are water-based solutions that have additive salts or other chemicals or components that alter the freezing temperature in precise, repeatable ways. It will be obvious to those skilled in the art that not all phase change materials are water-based, and that the actual choice of material may vary with no loss of novelty regarding the present disclosure.

Variation implementations of preferable embodiments may utilize alternate configurations of phase change materials. For example, multiple phase change materials may be mixed together in a single packet or pod. Alternatively, one packet or pod may be configured with multiple chambers to house multiple phase change materials. This configuration would allow for multiple stages of energy transfer between the phase change material packets and the gel material.

Other variation implementations of thermal packs may contain multiple phase change materials housed in different packets or pods. Packets or pods would not necessarily need to be the same size or shape, or be present in the identical number or weight to have a potentially beneficial impact on thermal pack performance. With reference to FIG. 5, a cross-sectional view of a thermal pack with two types of phase change material packets 503 and 504 is shown in (a). In this configuration, the two types of phase change packs are distributed randomly or quasi-randomly throughout the gel material 502. Alternate implementations may include a thin membrane, mesh, or other suitable barrier 505 within the lumen of the pouch to separate the two or more types of phase change materials. This may be advantageous in certain desired use cases. For example, in the case of medical applications, it may be useful as a safety precaution to keep a phase change material with a lower temperature in the upper compartment, further from patient skin contact.

With reference to FIG. 5, further variations of preferable embodiments may use additional layers. For example, in (c), a bottom (surface contacting side) layer 506 is added to the pack. This could be for comfort, to absorb moisture, for insulation, for infection control, for additional reasons, or for some combination of reasons. In (d), several additional layers are present in the design, including a top (non-surface contacting side) insulating layer 507 constructed of foam or another suitable material to enhance the thermal properties of the pack while at the same time shielding a user for unnecessary exposure to hot or cold energy on the side of the pack not intended for contact. Additionally, the pack in (d) includes a total covering sleeve 508 that is secured around the pack and can be removed. In some embodiments, this sleeve is reusable or disposable and is optional for use.

Preferable embodiments of methods for use will vary slightly depending upon the intended target application of the thermal pack. A cold pack with human-use applications intending to cool tissue to a temperature approaching the freezing point of water (for example, target temperatures in the 34-35 degree Fahrenheit range) may be used as an illustrative example. For this application, preferable embodiments may utilize phase change materials with freezing points a few degrees colder than the target temperature, for example in the 30-32 degree Fahrenheit range. Cold packs would be stored in a freezer or other suitable cooling chamber prior to use. Storage temperatures may vary depending on the exact gel-type (or equivalent) and phase change material formulations used. In some implementations of the preferred embodiment, storage temperatures may range from −15 to 0 degrees Fahrenheit. In alternate implementations storage temperatures may be warmer, closer to the freezing temperature of the phase change material in the embodiment of the devices and systems selected for use. Cold packs are removed from cold storage in a reasonable time period, for example 10-15 minutes or less, prior to application to skin. Cold packs are placed on the skin, conforming about curved surfaces (such as an arm or a leg) as necessary. Even at use-temperature, cold packs bend easily around these surfaces without sizeable gaps between contact points. For example, any gaps present should be small (less than one inch in any dimension) and the number of incidental small gaps should be low.

Preferable embodiments will exhibit superior performance relative to thermal packs that are currently commercially-available and other technologies described in the prior art. An illustrative example using test data from one preferable embodiment is shown in FIG. 6. In this test, a preferable embodiment of the presently disclosed thermal pack, configured as a cold pack, was benchmarked against a high-end commercially-available soft-gel cold pack described in the prior art. As shown in the figure, the presently-disclosed cold pack technology lowers the temperature of the target surface quickly and to a lower target temperature than the prior art pack. It is also able to maintain the target temperature over the 50 minute testing period. The prior art cold pack is unable to achieve a low temperature and is unable to maintain consistent cooling for an extended period of time. Both tested packs were stored/cooled under the same conditions, were of similar size and volume, were applied to the same thermal testing load, and contained gel-like materials with identical thermal and mechanical properties. As such, all variables aside from the novel features described in this disclosure were controlled, and performance improvements can be attributed solely to the novel multi-material construction devices, systems, and methods and other inventive aspects that are presently-described. These thermal performance achievements were achieved while maintaining the conformability, even coverage, and reusability features that are desirable. No technology described in the prior art is capable of simultaneously achieving all of these performance features.

### DESCRIPTION OF THE DRAWINGS

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

As shown in FIG. 1, selected characteristics of cold pack technologies described in the prior art and those associated with the presently-disclosed technology. ✓ indicates that a technology achieves performance requirement, X indicates that a technology does not achieve performance requirement, and ✓/X indicates that a technology may or may not achieve the requirement depending on certain factors, such as how the technology is configured. The key for performance requirements in the table is as follows: (a) can produce target temperatures in human tissue close to the freezing temperature of water, (b) doesn't over-shoot target temperature, (c) reaches target temperature quickly, (d) holds the desired temperature for an extended period of time, (e) stays mechanically conformable to a curved surface, (f) avoids/minimizes ‘gaps’ or regions of poor contact/thermal transfer, (g) can be regenerated quickly into a form that is ready for re-use.

As shown in FIG. 2, example preferable embodiments of the presently-disclosed devices and systems. In 2(a), a rectangular shaped thermal pack is shown. In 2(b), circular or ovular shaped thermal pack is shown. In (c), an example embodiment of a thermal pack 201 is shown to be conforming around the curved surface of a human leg 202.

As shown in FIG. 3, cross-sectional views of example embodiments of the devices, systems, and methods disclosed herein. In (a), a cold pack is shown in its room temperature state. The cold pack includes a pouch material 301 which encloses multiple constituent materials, including a gel-based material 302 and packets of phase-change material 303. Phase change material packets are dispersed randomly or quasi-randomly through the gel material. In this room temperature configuration, the material within packets 303 remains in a liquid state. In (b), the same cold pack shown after it has been stored at the appropriate temperature and is ready to use. At this temperature, material within phase change packets 303 has frozen into a solid state, symbolized in the drawing by the unfilled rectangles representing the packets. In (c), the cold pack 304 is shown at its intended use temperature conforming about a surface with a radius of curvature 305 without any gaps or wrinkles/buckles that inhibit thermal coupling.

As shown in FIG. 4, various examples of preferable embodiments for packets or pods that contain phase change material. In (a), top and side/oblique views of a rectangular “pillow” or “ravioli” configuration is shown. In (b), a cross-sectional view of a spherical or nearly-spherical pod 401 containing phase change material 402 in the liquid state is shown. In (c), a top view is shown of elongated compartments of phase change material 403 that are separated by an intermediary zone 404. In (d) and (e), top views are shown of grids of individual compartments 405 of phase change material arranged in orderly/symmetric and offset/asymmetric configurations, respectfully. In (f), a top view of linear arrangements of compartments of phase change material 405 with intermediary zones of material 406.

As shown in FIG. 5, cross-sectional views of variations of preferable embodiments of the devices and systems disclosed. In (a), a thermal pack with multiple types of phase change materials 503 and 504 distributed randomly throughout a gel material matrix 502 contained within an outer pouch 501 is shown. The two different types of phase change packets shown have different shape and may have different thermal and other material properties. In (b), an embodiment of the thermal pack which contains a partition 505 which separates multiple types or distributions of phase change material packets into distinct zones. In (c), a thermal pack which has specially-configured bottom layer 506. In (d), a thermal pack with a specially-configured top layer 507, shown enclosed in a protective sleeve 508.

As shown in FIG. 6, test data showing a performance comparison of a high-end commercially-available soft-gel cold pack described in the prior art vs. a cold pack embodiment of the present disclosure. As shown, the presently-disclosed cold pack technology lowers the temperature of the target surface quickly and to a lower target temperature than the prior art pack. It is also able to maintain the target temperature over the 50 minute testing period. The prior art cold pack is unable to achieve a low temperature and is unable to maintain consistent cooling for an extended period of time. Both tested packs were stored/cooled under the same conditions, were of similar size and volume, were applied to the same thermal testing load, and contained gel-like materials with identical thermal and mechanical properties. As such, all variables aside from the novel features described in this disclosure were controlled, and performance improvements can be attributed solely to the novel multi-material construction devices, systems, and methods and other inventive aspects that are presently-described.

What is claimed is:

1. A thermal pack adapted for use in a muscle stimulation system, comprising:
an external housing with a first material therein and at least one phase change element therein.
